(19) United States
(12) Patent Application Publication
(10) Pub. No.: US 2009/0074742 A1
(43) Pub. Date: Mar. 19, 2009

(54) PROTEIN, OSTEOCLAST DIFFERENTIATION INHIBITOR, INFLAMMATORY BONE RESORPTION THERAPEUTIC AGENT, GENE, RECOMBINANT VECTOR, METHOD OF MANUFACTURING A PROTEIN, METHOD OF INHIBITING OSTEOCLAST DIFFERENTIATION, AND METHOD OF TREATING INFLAMMATORY BONE RESORPTION

(75) Inventor: Yasuhiro Kobayashi, Nagano (JP)

Correspondence Address: LADAS & PARRY, 5670 WILSHIRE BOULEVARD, SUITE 2100, LOS ANGELES, CA, 90036-5679, US

(73) Assignee: MATSUMOTO DENTAL UNIVERSITY, Nagano (JP)

(21) Appl. No.: 12/175,217

(22) Filed: Jul. 17, 2008

(30) Foreign Application Priority Data
Sep. 14, 2007 (JP) 2007-240015

(51) Int. Cl. C07K 14/435; C12N 15/12; C12N 15/54; C12P 21/02; C12N 5/06; C12N 15/85; A61K 38/17; A61K 38/45; A61P 19/00

(52) U.S. Cl. 424/94.5; 530/350; 536/23.5; 435/69.1; 435/377; 435/193; 536/23.2; 435/320.1; 514/12

(57) ABSTRACT

There are provided a protein which comprises the amino acid sequence of an extracellular region of Ror2 and is water-soluble, an osteoclast differentiation inhibitor for inhibiting differentiation of a precursor cell of osteoclast into an osteoclast, which comprises the protein as described above, an inflammatory bone resorption therapeutic agent for treating inflammatory bone resorption, which comprises the protein as described above, a gene which codes a protein comprising the amino acid sequence of an extracellular region of Ror2 and being water-soluble, a recombinant vector which comprises the gene as described above, a method of manufacturing a protein, which comprises a step of causing expression of a gene coding a protein comprising the amino acid sequence of an extracellular region of Ror2 and being water-soluble in a microorganism to synthesize the protein and a step of extracting the protein from the microorganism into water or an aqueous solution, a method of inhibiting osteoclast differentiation, which uses the protein as described above, and a method of treating inflammatory bone resorption, which uses a protein as described above.

### BACKGROUND OF THE INVENTION

1. Field of the Invention

The present invention relates to a protein, an osteoclast differentiation inhibitor, an inflammatory bone resorption therapeutic agent, a gene, a recombinant vector, a method of manufacturing a protein, a method of inhibiting osteoclast differentiation, and a method of treating inflammatory bone resorption.

2. Description of the Related Art

As disclosed in Ryumachi, Vol. 43(4), pp. 624-631, 2003 (in Japanese), for example, rheumatoid arthritis is an autoimmune, chronic and inflammatory disease wherein proliferated pannus penetrate into a bone actively so as to cause multiple articular destructions. Due to the development of non-steroidal anti-inflammatory agents and drugs acting on immunoregulation principally, such as steroids, these medicines have been reported to control the pain and inflammation in a certain level. However, no therapy for preventing bone resorption completely has yet been established at present.

Furthermore, as disclosed in Clinical Immunology, Vol. 44(6), pp. 622-628, 2005 (in Japanese), bone resorption caused by osteoclasts and osteogenesis caused by osteoblasts are repeated even after bone is formed, thereby keeping the concentration of calcium in body fluid. Osteoclast differentiation and activation which contribute to bone resorption are regulated by osteoblasts strictly. That is, the osteoblast expresses a receptor activator of nuclear factor (NF)-κB ligand (RANKL) and a macrophage-colony stimulating factor (M-CSF), which are important cytokines for osteoclast differentiation and activation. The RANKL is secreted by stimulation of a bone resorption factor such as 1α,25-dihydroxy vitamin D₃. On the other hand, the M-SCF is secreted from an osteoblast constitutively.

Moreover, it has been known from Clinical Immunology, Vol. 44(6), pp. 641-646, 2005 that T-cells invading into an inflammatory synovial tissue yield RANKL, and thereby, promote osteoclast differentiation for bone resorption in rheumatoid arthritis. Furthermore, a tumor necrosis factor-α (TNF-α) that is one of inflammatory cytokines also induces expression of RANKL in osteoblasts. Then, TNF-α directly acts on precursor cells of osteoclasts, and thereby, induces osteoclast differentiation. For this reason, an antibody against RANKL or TNF-α has been applied for inhibiting bone resorption in rheumatoid arthritis. However, because the RANKL and TNF-α are also important cytokines in a normal immunoreaction, there are several issues regarding the suppression of normal immunoreaction by these antibodies.

Moreover, the Journal of Clinical Investigation, Vol. 116, No. 5, 2006, pp. 1202-1209 discloses matters as described below.

Wnt is a cytokine having various biological activities from organogenesis to carcinoma development in an ontogenetic process. The Wnt is a secretory glycoprotein. The Wnt signaling pathways are generally classified into two pathways, that is, a canonical pathway that is mediated by β-catenin and a non-canonical pathway which is not mediated thereby.

When the Wnt binds to a receptor, the β-catenin, a transcriptional co-activator, is accumulated in cytoplasm and translocated to a cell nucleus. The β-catenin binds to the promoter region of a target gene together with TCF and LEF, transcription factors, and initiates mRNA synthesis of the gene. LiCl, an inhibitor for GSK-3β, activates the canonical pathway.

In the non-canonical pathway, c-jun N-terminal kinase (JNK), calmodulin dependent kinase (CaNMK), protein kinase C (PKC), and the like are activated. Wnt5a and Ror2, a co-receptor of Wnt5a, activate the non-canonical signaling pathway.

Studies of mutations of LRP5 in a human being or mouse have shown that osteoblast differentiation or osteogenesis is activated when the canonical pathway of the Wnt is activated.

On the other hand, it has been shown that the expression of RANKL is reduced when the canonical pathway is activated. Furthermore, it has also been shown that when β-catenin gene is deleted in a matured osteoblast, the expression of osteoprogerin, which is an inhibition factor of the RANKL, is reduced and bone resorption is enhanced so that a bone mass is reduced.

Moreover, Arthritis & Rheumaism, Vol. 44, No. 4, 2001, pp. 772-781, Rheumatology, Vol. 44, 2005, pp. 708-713, PNAS, Vol. 97, No. 6, 2000, pp. 2791-2796, etc., disclose that the expression of Wnt5a is caused very frequently in a synovial tissue sampled from a rheumatic patient. Also, it has been reported that the expression of IL-15 or RANKL, which are inflammatory cytokines, was enhanced when a cell (synovial cell) obtained from a synovial tissue was stimulated by the Wnt5a in vitro. However, the role of the Wnt5a in rheumatoid bone destruction has been yet unclear because no method capable of blocking the action of the Wnt5a efficiently has been established.

In addition, Genes to Cells, 2003, pp. 645-654 has reported that Wnt5a binds to a cysteine-rich domain (CRD) of the extracellular region of Ror2 whereby a tyrosine kinase region in the intracellular region of the Ror2 is activated so as to mediate a signal.

As described above, there is a method for administrating an anti-inflammatory agent or antibody for an inflammatory cytokine is provided for a therapy of rheumatoid arthritis at present. However, the therapy with an anti-inflammatory agent could suppress inflammation but has not yet led to enable to suppress bone resorption completely. Also, for the therapy with an antibody for TNF-α or RANKL, there are some issues for the possibility of causing reduction of normal immunoreaction, because the TNF-α or the RANKL is a cytokine which also plays an important role in the normal immunoreaction.

In such a situation, the inventor has found a method for reducing or suppressing bone resorption without particularly affecting an immune system, so as to provide a novel protein, osteoclast differentiation inhibitor, inflammatory bone resorption therapeutic agent, gene, recombinant vector, and method of manufacturing a protein.

### SUMMARY OF THE INVENTION

According to one aspect of the present invention, there is provided a protein which comprises the amino acid sequence of an extracellular region of Ror2 and is water-soluble.

According to another aspect of the present invention, there is provided an osteoclast differentiation inhibitor for inhibiting differentiation of a precursor cell of osteoclast into an osteoclast, which comprises the protein as described above.

According to another aspect of the present invention, there is provided an inflammatory bone resorption therapeutic agent which comprises the protein as described above.

According to another aspect of the present invention, there is provided a gene which codes a protein comprising the amino acid sequence of an extracellular region of Ror2 and being water-soluble.

According to another aspect of the present invention, there is provided a recombinant vector which comprises the gene as described above.

According to another aspect of the present invention, there is provided a method of manufacturing a protein, which comprises a step of causing expression of a gene coding a protein comprising the amino acid sequence of an extracellular region of Ror2 and being water-soluble in a microorganism so as to synthesize the protein and a step of extracting the protein from the microorganism into water or an aqueous solution.

According to another aspect of the present invention, there is provided a method of inhibiting osteoclast differentiation, which uses the protein as described above.

According to another aspect of the present invention, there is provided a method of treating inflammatory bone resorption, which uses a protein as described above.

### BRIEF DESCRIPTION OF THE DRAWINGS

FIG. 1 is a diagram that schematically illustrates an example of a protein according to an embodiment of the present invention.

FIG. 2 is a graph showing the effect of Wnt5a on the differentiation of a bone marrow macrophage into osteoclasts.

FIG. 3 is a graph showing the effect of Wnt5a on the differentiation of a bone marrow macrophage into osteoclasts with a suppressed expression of a protein Ror2.

FIG. 4 is a graph showing the effect of a soluble Ror2 on osteoclast differentiation in a co-culture system of bone marrow cells and osteoblasts.

FIG. 5 is a graph showing the effect of soluble Ror2 administration on the bone resorption of a tarsal of a rheumatoid arthritis model mouse.

FIG. 6 is a graph showing the effect of soluble Ror2 administration on the content of a cancellous bone at the proximal end of the tibia of a rheumatoid arthritis model mouse.

FIG. 7 is a graph showing the effect of soluble Ror2 administration on the number of osteoclast(s) at the proximal end of the tibia of a rheumatoid arthritis model mouse.

### DETAILED DESCRIPTION OF THE PREFERRED EMBODIMENTS

Next, illustrative embodiments of the present invention are described with reference to the drawings.

A first embodiment of the present invention is a protein characterized by comprising the amino acid sequence of an extracellular region of Ror2 and being water-soluble.

Herein, the term “Ror2” means a publicly-known receptor-type tyrosine-kinase-like orphan receptor 2 which may be expressed in a precursor cell of an osteoclast. Furthermore, an extracellular region of Ror2 consists of an immunoglobulin-like domain (IgLD) of Ror2, a cysteine-rich domain (CRD) of Ror2, and a kringle domain (KD) of Ror2 in order from the outside of the receptor Ror2. In addition, because each of the amino acid sequences of IgLD, CRD and KD which constitute an extracellular region of Ror2 is known publicly, the amino acid sequence of an extracellular region of Ror2 is also known publicly. However, a protein comprising the amino acid sequence of an extracellular region of Ror2 and being water-soluble has not been known yet. Moreover, a protein according to the first embodiment of the present invention is not particularly limited as far as it comprises the amino acid sequence of an extracellular region of Ror2 and is water-soluble.

A protein according to the first embodiment of the present invention may preferably be a protein characterized by further comprising the amino acid sequence of GST.

Herein, the term “GST” means a glutathione S-transferase. The amino acid sequence of GST is also known publicly. Furthermore, a protein according to the first embodiment of the present invention may be a protein characterized by consisting of the amino acid sequence of an extracellular region of Ror2 and the amino acid sequence of GSR and being water-soluble.

A second embodiment of the present invention is an osteoclast differentiation inhibitor for differentiation of a precursor cell of osteoclast into an osteoclast, characterized by comprising a protein according to the first embodiment of the present invention.

Herein, a protein according to the first embodiment of the present invention may make it possible to inhibit differentiation of a precursor cell of osteoclast into an osteoclast and may also make it possible to use the protein according to the first embodiment of the present invention as an active ingredient of a drug for inhibiting differentiation of a precursor cell of osteoclast into an osteoclast. Furthermore, a content (or concentration) of a protein according to the first embodiment of the present invention which is contained in an osteoclast differentiation inhibitor according to the second embodiment of the present invention may preferably be 100 μg/ml or more and 1 mg/ml or less.

A third embodiment of the present invention is an inflammatory bone resorption therapeutic agent for treating inflammatory bone resorption, characterized by comprising a protein according to the first embodiment of the present invention.

Herein, it may be possible to use a protein according to the first embodiment of the present invention as an active ingredient of a drug for treating inflammatory bone resorption because the protein according to the first embodiment of the present invention may make it possible to inhibit differentiation of a precursor cell of osteoclast into an osteoclast. An inflammatory bone resorption therapeutic agent according to the third embodiment of the present invention may comprise water or an aqueous solvent for dissolving a protein according to the first embodiment of the present invention. The aqueous solvent may be, for example, a phosphate buffer. A disease of inflammatory bone resorption may include, for example, a symptom of bone resorption in a rheumatoid arthritis. Furthermore, a content (or concentration) of a protein according to the first embodiment of the present invention which is contained in an inflammatory bone resorption therapeutic agent according to the third embodiment of the present invention may preferably be 100 μg/ml or more and 1 mg/ml or less.

A fourth embodiment of the present invention is a gene characterized by coding a protein comprising the amino acid sequence of an extracellular region of Ror2 and being water-soluble.

A gene according to the fourth embodiment of the present invention may preferably be a gene characterized in that the protein comprises the amino acid sequence of GST. Furthermore, the protein may be a protein characterized by consisting of the amino acid sequence of an extracellular region of Ror2 and the amino acid sequence of GST being water-soluble.

A fifth embodiment of the present invention is a recombinant vector characterized by comprising a gene according to the fourth embodiment of the present invention.

Herein, a recombinant vector according the fourth embodiment of the present invention may be obtained by inserting a DNA fragment encoding the fourth embodiment of the present invention into a vector. The vector may include, for example, a plasmid DNA of Escherichia coli (E. coli) and the like. For example, it may be possible to synthesize a protein comprising the amino acid sequence of an extracellular region of Ror2 and being water-soluble in the fourth embodiment of the present invention by introducing a recombinant vector according to the fourth embodiment of the present invention into a cell of a microorganism so as to cause expression of a gene according to the fourth embodiment of the present invention in the microorganism.

A sixth embodiment of the present invention is a method of manufacturing a protein, characterized by comprising a step of causing expression of a gene coding a protein comprising the amino acid sequence of an extracellular region of Ror2 and being water-soluble in a microorganism so as to synthesize the protein and a step of extracting the protein from the microorganism into water or an aqueous solution.

Herein, the microorganism may include, for example, Escherichia coli (E. coli), a cultured cell of a mammal or insect, and the like. In order to cause expression of a gene coding a protein comprising the amino acid sequence of an extracellular region of Ror2 and being water-soluble in a microorganism, for example, a recombinant vector may be used which comprises a gene coding a protein comprising the amino acid sequence of an extracellular region of Ror2 and being water-soluble.

A method of manufacturing a protein according to the sixth embodiment of the present invention may preferably be a method of manufacturing a protein characterized in that the protein comprises the amino acid sequence of GST and the aqueous solution comprises a glutathione. The protein may be a protein characterized by consisting of the amino acid sequence of an extracellular region of Ror2 and the amino acid sequence of GST and being water-soluble. Furthermore, the glutathione may preferably be a reduced-form of glutathione.

When the protein comprises the amino acid sequence of GST and the aqueous solution comprises a glutathione, the GST may better couple with the glutathione and accordingly it may be possible to extract (or purify) the protein from a microorganism more efficiently. Furthermore, when the glutathione is a reduced form of glutathione, it is considered that the safety thereof for a living organism with respect to use of an aqueous solution in which the protein has been extracted is relatively high because a reduced-form of glutathione is a principal glutathione existing inside a cell.

A seventh embodiment of the present invention is a method of inhibiting osteoclast differentiation characterized by using a protein according to the first embodiment of the present invention. In a method of inhibiting osteoclast differentiation according to the seventh embodiment of the present invention, for example, an osteoclast differentiation inhibitor according to the second embodiment of the present invention may be used.

An eighth embodiment of the present invention is a method of treating inflammatory bone resorption characterized by using a protein according to the first embodiment of the present invention. In a method of treating inflammatory bone resorption according to the eighth embodiment of the present invention, for example, an inflammatory bone resorption therapeutic agent according to the third embodiment of the present invention may be used.

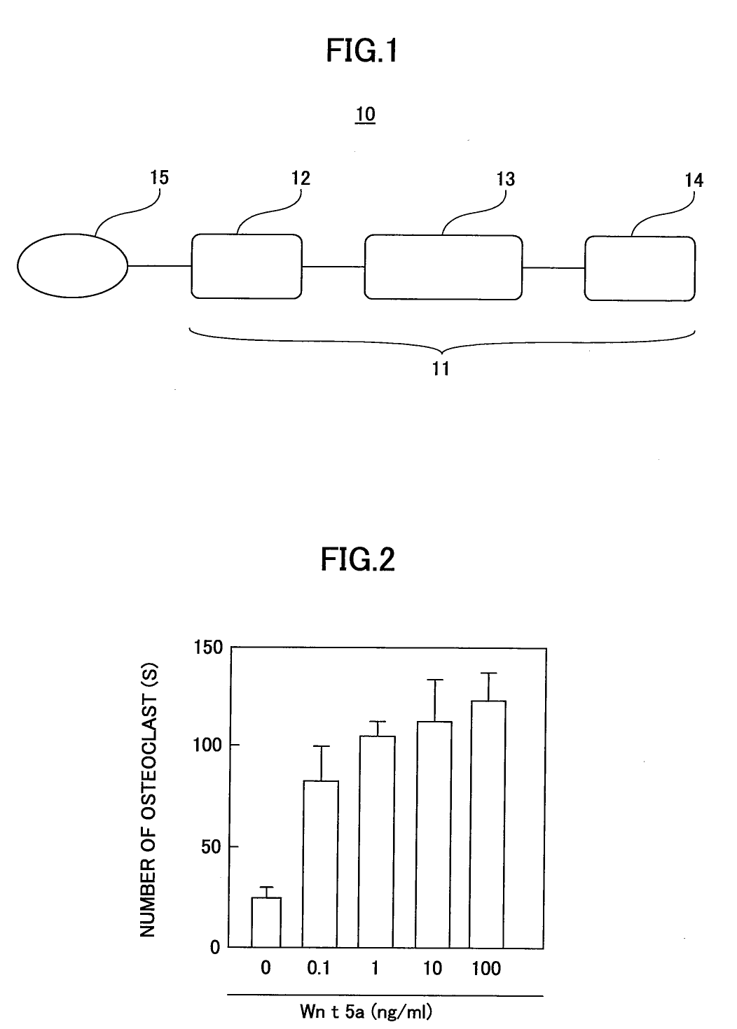

FIG. 1 is a diagram that schematically illustrates an example of a protein according to an embodiment of the present invention.

A protein (10) as shown in FIG. 1 is composed of the amino acid sequence of an extracellular region of Ror2 (11) and the amino acid sequence of GST (15). Therefore, the protein (10) is water-soluble, and for example, may be dissolved in a phosphate buffer (aqueous solution). The amino acid sequence of an extracellular region of Ror2 (11) is composed of the amino acid sequence of an immunoglobulin-like domain (IgLD) of Ror2 (12), the amino acid sequence of a cysteine-rich domain (CRD) of Ror2 (13), and the amino acid sequence of a kringle domain (KD) of Ror2 (14). That is, the amino acid sequence of GST (15), the amino acid sequence of IgLD of Ror2 (12), the amino acid sequence of CRD of Ror2 (13), and the amino acid sequence of KD of Ror2 (14) are linked linearly in this order in the protein (10).

Specific embodiments of the present invention are described in detail below but the present invention should not be limited to them.

A specific embodiment of the present invention indicates that Wnt5a would play an important role for osteoclast differentiation in bone resorption, provides to produce a soluble Ror2 (sRor2) which is an inhibitor for Wnt5a, and provides a method of controlling progression of bone resorption by inhibiting the activity of Wnt5a.

More particularly, a specific embodiment of the present invention aims to elucidate that a Wnt5a signaling pathway which is not mediated by β-catenin promotes osteoclast differentiation in regard to bone resorption in rheumatoid arthritis. Then, there is provided a method of preventing progression of bone resorption in which a soluble Ror2 (sRor2) that is an inhibitor for Wnt5a is prepared and administrated into, for example, a rheumatoid arthritis model mouse.

Prior to descriptions of a preferred embodiment of the present invention, the role of Wnt in maintenance of bone mass is previously described.

As described above, the Wnt is a secretory glycoprotein, whose cell activation signaling pathway is generally classified into two pathways, that is, a canonical pathway that is mediated by β-catenin and a non-canonical pathway that is not mediated thereby.

Onset of pseudoglioma with osteoporosis is caused by abnormality of LRP5 which is a coreceptor activating the canonical pathway mediated by β-catenin. It has been shown that osteoblast differentiation or bone formation is reduced due to abnormality of LRP5 so as to reduce bone mass. These reports indicate that osteoblast differentiation or bone formation is activated when the canonical pathway of the Wnt is activated.

The role of the canonical pathway of the Wnt in bone resorption has been analyzed. It has been shown that when the β-catenin gene is deleted in a matured osteoblast, expression of osteoprogerin is reduced and bone resorption is enhanced so as to reduce bone mass. This result means that expression of the osteoprogerin increases and osteoclast differentiation and activation are suppressed when the canonical pathway of the Wnt is activated in an osteoblast.

However, it has not been clear what influences the non-canonical signal of the Wnt which may directly act on a precursor cell of osteoclast or an osteoclast has on bone resorprion.

Herein, a specific embodiment of the present invention aims to elucidate that Wnt5a promotes osteoclast differentiation. More specifically, a soluble protein of Ror2 (soluble Ror2) which is an inhibitor for the Wnt5a is prepared in order to inhibit the action of the Wnt5a and used as a therapeutic agent for rheumatoid arthritis which contains the prepared soluble Ror2.

Next, some examples of a preferred embodiment of the present invention are specifically described with reference to the drawings. FIG. 2 to FIG. 7 show the results of experiments for studying that a soluble Ror2 suppresses osteoclast differentiation, by using cultivation and a rheumatoid arthritis model mouse.

First, a method of differentiation of a macrophage into an osteoclast in vitro is described below. Bone marrow cells were sampled from the long tubular bones of legs of 6 to 8 week-old mice. The isolated bone marrow cells were cultured for 3 days under the presence of M-CSF that was a macrophage-stimulating factor so as to promote their differentiation into macrophages. These cells are referred to as bone marrow macrophages. Then, the bone marrow macrophages were further stimulated by the M-CSF and RANKL so that they were differentiated into osteoclasts.

Next, the effect of Wnt5a on differentiation of a macrophage into an osteoclast is described below. Wnt5s was added into the culture system in order to induce differentiation of the bone marrow macrophages into osteoclasts. FIG. 2 shows the results of the culture conducted with addition of Wnt5a in the culture experiment of differentiation of the macrophages into osteoclasts due to the M-CSF and the RANKL. As shown in FIG. 2, osteoclast differentiation was promoted dose-dependently in the culture with addition of Wnt5a. In other words, the Wnt5a promoted the differentiation of the bone marrow macrophages into osteoclasts, depending on the amount of the added Wnt5a. As a cell receptor for Wnt5a is described herein, it is known that the Wnt5a binds to Frizzled 2 or Frizzeled 5 causing expression of a cell, activates the cell, and also binds to Ror1 or Ror2 which is a coreceptor. In the present example, the bone marrow macrophages that were precursor cells of osteoclast caused expressions of mRNAs of Frizzled 2, Frizzled 5 and Ror2 which had been reported as receptors for the Wnt5a.

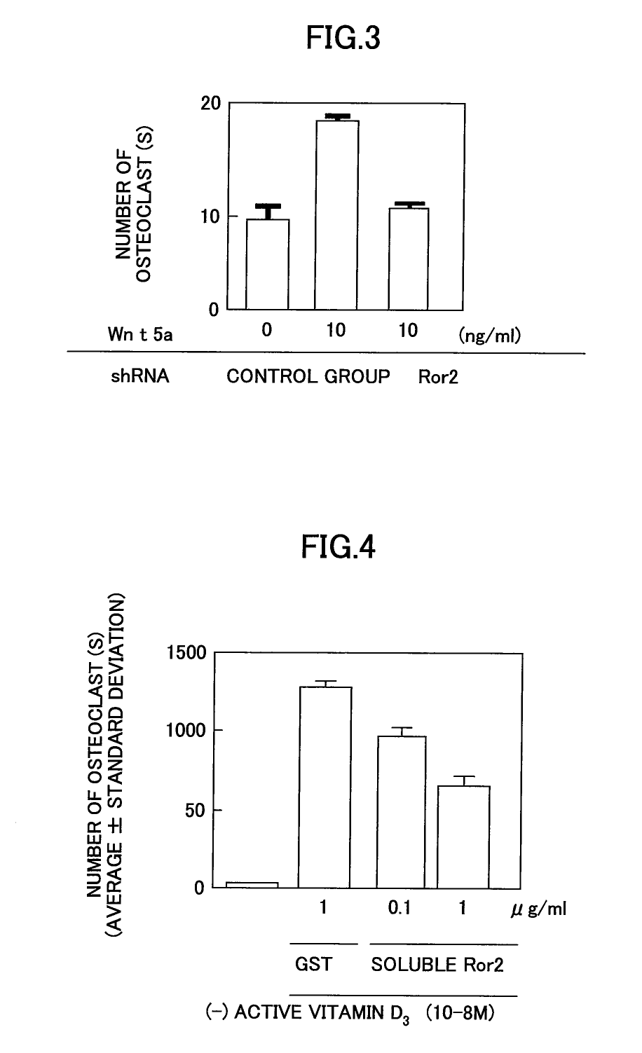

Then, a study was conducted with respect to whether Ror2 was essential for Wnt5a to have effect of promoting osteoclast differentiation. The results are shown in FIG. 3. Specifically, expression of Ror2 of the bone marrow macrophage was reduced by using shRNA. Because no effect of Wnt5a on promotion of osteoclast differentiation was observed for the bone marrow macrophage with reduced Ror2, it was suggested that the effect of the Wnt5a on promotion of osteoclast differentiation would be mediated by the Ror2.

It is also known that Wnt5a binds to a cysteine-rich domain (CRD) of an extracellular region of Ror2 and the binding activates a tyrosine kinase domain of the intracellular region of the Ror2 so as to transfer a signal into the inside of a cell.

Herein, a soluble Ror2 was prepared which could bind to Wnt5a but could not transfer a signal into the inside of a cell in the present example.

Next, a soluble Ror2 (receptor protein) and a manufacturing method thereof in the example of the present invention are described below.

A vector containing the cDNA of a receptor Ror2 (Clone No. 30535615) was available from Open Biosystems. A DNA fragment coding the extracellular region of the Ror2 was amplified by means of a polymeraze chain reaction (PCR) method while the cDNA of the receptor Ror2 was template. The amplified cDNA of the extracellular region of the Ror2 was connected to the downstream of a gene of glutathione-S-transferase (GST) which was available from GE Healthcare Biosciences, namely, a vector pGEX-4T-2 having a gene of GST. The vector having the gene of the extracellular region of GST-Ror2 was introduced into an Escherichia coli (E. coli) and the Escherichia coli in a Luria-Bertani (LB) culture medium containing 100 μg/ml of ampicillin was incubated at 37° C. overnight. 20 ml of the cultured liquid of the Escherichia coli was inoculated to 400 ml of another LB culture medium containing 100 μg/ml of ampicillin. After the liquid of Escherichia coli was incubated at 37° C. for 2 hours, isopropyl-β-D-(−)-thiogalactopyranoside (IPTG) with a concentration of 1 nM which was a protein synthesis inductor was added so as to induce protein synthesis. After addition of IPTG, incubation was conducted at 37° C. for 6 hours such that the Escherichia coli synthesized a conjugated protein of GST-soluble Ror2.

The Escherichia coli causing expression of a conjugated protein of GST-soluble Ror2 (simply, a soluble Ror2) was fragmented by means of ultrasonic treatment using an ultrasonic disintegrator (Bioruptor UCD-200 from Cosmo Bio Co. Ltd.). A conjugated protein of GST-soluble Ror2 was purified from the fragmented Escherichia coli component by utilizing coupling of GST with glutathione. That is, the supernatant of the solution of the fragmented Escherichia coli (containing the conjugated protein of GST-soluble Ror2) was admixed with Sepharose beads to which glutathione was bound (Glutathione Sepharose FF from GE Healthcare Biosciences), at 4° C. for 16 hours, such that the conjugated protein of GST-soluble Ror2 was bound to the Glutathione Sepharose. Then, the conjugated protein of GST-soluble Ror2 bounded to the Glutathione Sepharose was recovered from the Escherichia coli component. That is, the Escherichia coli component which was non-specifically bounded to the Glutathione Sepharose FF was washed out with a phosphate buffer (PBS, 137 mM of NaCl, 10 mM of phosphate, 2.7 mM of KCl, and pH of 7.4) containing 0.5 wt % of Triton X-100 (available from SIGMA-ALDRICH Japan K.K.) so as to provide a condition that only the conjugated protein of GST-soluble Ror2 remains on the beads as far as possible. These beads were suspended in a solution containing an excess amount of the reduced form of glutathione (5 mM of the reduced form of glutathione (Wako Pharmaceutical Co., Ltd.), Tris buffer containing 150 mM of salt, and pH of 8.0) so that the conjugated protein of GST-soluble Ror2 was extracted from the Sepharose beads with the bound glutathione.

The solution of extracted soluble Ror2 was contained in a cassette of dialysis membrane and stirring was conducted in 3 liters of a PBS. This was repeated two times, whereby the solution component used for the extraction (Tris buffer containing 5 mM of the reduced form of glutathione and 150 mM of salt, and pH of 8.0) could almost completely be replaced with a PBS. That is, the buffer component used for the extraction, which contains a small amount of glutathione, was removed from the solution of soluble Ror2 by means of dialysis so that the solution became a PBS.

Endotoxin originating from the Escherichia coli which was contained in the solution of soluble Ror2 was removed by means of phase separation with Triton X-114 (available from SIGMA-ALDRICH Japan K.K.). That is, Triton X-114 was added into the solution of soluble Ror2 such that the final concentration thereof was 1 wt % and mixing was conducted at 4° C. for 30 minutes.

The mixed solution of soluble Ror2 was warmed at 37° C. so as to precipitate Triton X-114. This was centrifuged so as to deposit the Triton X-114. Herein, the endotoxin contained in the solution transferred into a precipitation phase together with the Triton X-114.

The supernatant was recovered and phase separation with the Triton X-114 was repeated 6 times.

The amount of the endotoxin in the solution from which the endotoxin should have been removed was quantified by means of gelation reaction of limulus lysate.

The solution of soluble Ror2 was used in which the amount of the endotoxin was 0.1 EU (endotoxin unit) or less per 1 μg of protein.

Next, a study was conducted with respect to whether the protein Wnt5a could bind to the prepared soluble Ror2.

The soluble Ror2 and the protein Wnt5a were admixed into a PBS buffer (phosphate buffer). When the soluble Ror2 was recovered from this solution by using Sepharose beads to which the glutathione was bound, the Wnt5a bound to the soluble Ror2 was recovered together. However, the Wnt5a could not be recovered from the solution in which GST and the protein Wnt 5a were admixed into a PBS buffer. Therefore, it was confirmed that the soluble Ror2 bound to the Wnt5a.

Next, a study was conducted with respect to whether osteoclast differentiation caused by Wnt5a could be suppressed by the prepared soluble Ror2. The results are shown in FIG. 4. The effect of promoting osteoclast differentiation in the culture in which Wnt5a was added was found. When the soluble Ror2 was thus added, the effect of promoting osteoclast differentiation caused by the Wnt5a was suppressed. From these results, it was shown that the prepared soluble Ror2 bound to the Wnt5a so as to inhibit the action of the Wnt5a.

Usually, macrophages which are precursor cells of osteoclast, contact ostoblasts and are stimulated by RANKL expressed by the osteoblasts, whereby osteoclast differentiation is caused. When osteoblasts obtained from a cranial bone of a mouse and bone marrow cells from a long tubular bone thereof were cultured together and an active vitamin D₃ as a bone resorption stimulating factor was added therein, osteoclast differentiation was caused more close to the in-vivo condition, even in such a cell culture experiment. The results of addition of the soluble Ror2into this osteoclast differentiation-culture system are shown in FIG. 4. When the soluble Ror2 was added, osteoclast differentiation which was induced by addition of the active vitamin D₃ was suppressed dose-dependently.

As expression of the Wnt5a in osteoblasts was studied, significant expression of the Wnt5a was found in the osteoblasts, compared to the precursor cells of osteoclasts. That is, it was shown that the osteoblasts constantly secreted the Wnt5a and the Wnt5a secreted from the osteoblasts stimulated the precursor cells of osteoclasts together with the RANKL so as to promote the osteoclast differentiation. Furthermore, it was also shown that the soluble Ror2 could block the Wnt5a signal and suppress the osteoclast differentiation.

Meanwhile, it has been reported that a high expression of Wnt5a is found in a rheumatoid synovial membrane. However, the role of Wnt5a in the pathology of rheumatism has not been elucidated yet.

Therefore, the purified soluble Ror2 as described above was administered to a culture system in which osteoclast differentiation was induced or a model in which in vivo bone resorption was enhanced.

Herein, a method for manufacturing a rheumatoid arthritis model mouse is described below. A monoclonal antibody for type-II collagen which was much contained in an articular tissue was administered at 3 mg per one body intravenously. After 2 days from administration of the antibody, a lipopolysaccharide (LPS) was administered at 75 μg per one body so as to cause onset of arthritis. After the onset of arthritis in rheumatoid arthritis model mice, the mice were classified into 4 groups with respect to administration of a saline, GST, or the soluble Ror2 (2 μg or 20 μg). Each drug was administered into the abdominal cavities of mice in each group every day for 2 weeks.

Herein, a method for administrating the soluble Ror2 is described. The dose of the soluble Ror2 was determined according to the following method. Osteoblasts and bone marrow cells were cultured together. Active vitamin D₃ was added into this culture system so as to induce osteoclasts. This culture system with the induced osteoclasts was diluted with a saline (0.9% NaCl) such that the final concentration of the soluble Ror2 was 500 ng/ml to 2 μg/ml. Then, the concentration of the soluble Ror2, which could most suppress osteoclast differentiation, was determined. 0.2 ml of the soluble Ror2 at a concentration which was 100 times that concentration was administered into the abdominal cavities of mice with a body weight of 30 g every day. The administration was conducted for 2 weeks.

First, one clinical symptom of rheumatoid arthritis is swelling of limbs. In regard to the swelling of limbs, no statistically significant difference was found among the 4 groups for which the saline, the GST and the soluble Ror2s (2 μg and 20 μg) were administered, respectively.

Then, the condition of bone resorption on a tarsal bone was observed by using a Micro-CT. For the control group in which no rheumatism was caused, no raduiolucent region (black portion in the bone) which indicated post-bone-resorption was found on a tarsal bone. For the group in which rheumatism was caused, many radiolucent regions were found on a tarsal bone. For the groups in which 20 μg of soluble Ror2 was administered, the amount of raduiolucent regions was reduced significantly. FIG. 5 shows the results of quantification of the raduiolucent regions of the bones in which rheumatism was caused. For the group in which 20 μg of the soluble Ror2 was administered, the amount of resorotion portions was significantly reduced, compared to the group of GST administration.

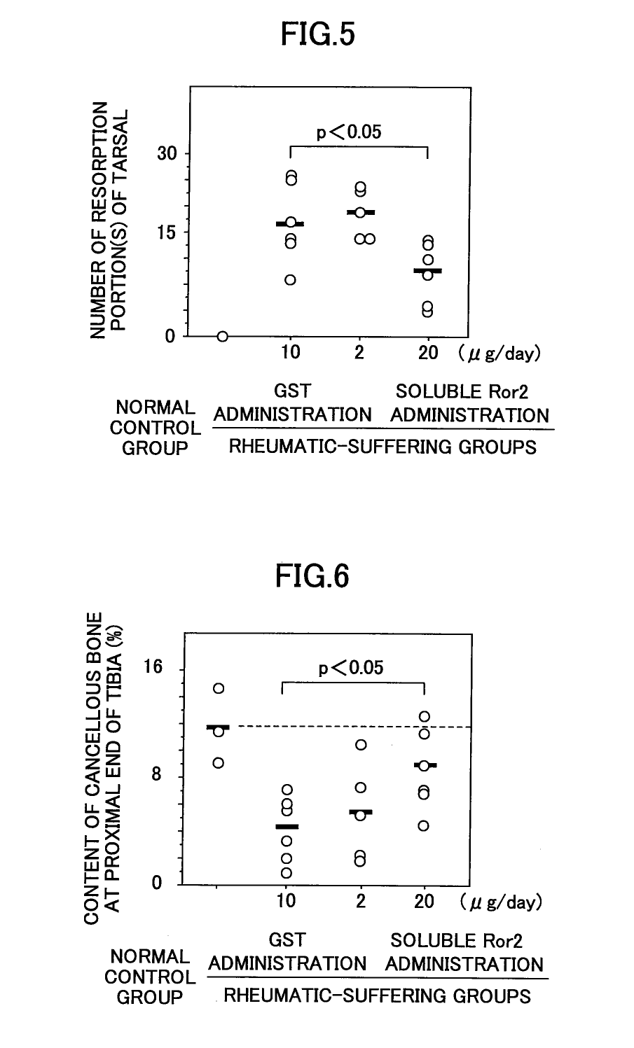

Then, the condition of proximal regions of a tibial bone was studied by using a micro-CT. FIG. 6 shows the results of quantification of the amount of a cancellous bone which were obtained by means of the micto-CT. A significant reduction of the cancellous bone was found in the group of GST administration in which onset of rheumatism was found, compared to the control group in which no onset of rheumatism was found. On the other hand, reduction of the cancellous bone which was caused by rheumatism was suppressed with respect to the mice to which 20 μg of the soluble Ror2 was administered.

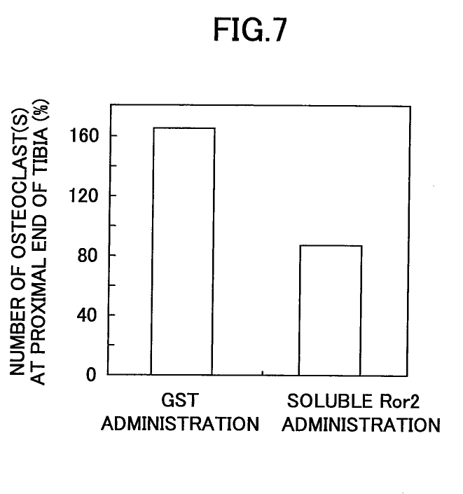

Then, a section of the tissue in the proximal region of the tibial bone was cut out and stained with a tartrate-resistant acid phosphatase (TRAP) which was a marker enzyme for an osteoclast. FIG. 7 shows the results of quantification of the number of osteoclasts found in the proximal region of the tibial bone. For the groups of administration of the soluble Ror2, the amount of TRAP-positive osteoclasts in the proximal region of the tibial bone was reduced significantly.

Thus, in the pathology of rheumatoid arthritis, it was demonstrated that Wnt5a yielded from a synovial tissue or an osteoblast was mediated by a receptor Ror2 existing on a macrophage, transferred a signal into the inside of a cell, and enhanced osteoclast differentiation together with RANKL in a coordinated manner so as to cause bone resorption.

Furthermore, administration of the soluble Ror2 against rheumatoid bone destruction blocked a signal of Wnt5a and suppressed osteoclast differentiation. From this mechanism, it was confirmed that the soluble Ror2 was to prevent bone resorption involved in rheumatism.

It is considered that at least one embodiment of the present invention could be available for an inhibitor of bone resorption using a Wnt receptor protein, the soluble Ror2, and inhibition of inflammatory bone resorption by means of a Wnt receptor protein, the soluble Ror2.

For example, no therapeutic agent for completely remedying a rheumatoid arthritis has been provided yet, while there is a drug such as an antirheumatic drug for suppressing pain in a symptomatic therapy with a drug and there is also an excisional operation for a patient with a worsen symptom. However, it is expected to be utilized as a therapeutic agent for a rheumatoid arthritis because some of the embodiments of the present invention could be utilized to adjust the balance between osteogenesis and osteoclasts.

Thus, it is expected that the soluble Ror2 could be used for treating various kinds of diseases resulting in bone reduction and serve to prevent the reduction of bone mass in the field of medical treatment. In particular, it is considered that it could serve as a therapeutic agent for therapy of a rheumatoid arthritis for which no assured therapy has been established yet.

Although the embodiment(s) and specific example(s) of the present invention have been specifically described above, the present invention is not limited to the embodiment(s) or specific example(s) and the embodiment(s) and specific example(s) of the present invention can be altered or modified without departing from the spirit and scope of the present invention.

The present application is based on Japanese priority application No. 2007-240015 filed on Sep. 14, 2007, the entire contents of which priority application are hereby incorporated by reference.

What is claimed is:

1. A protein comprising the amino acid sequence of an extracellular region of Ror2 and is water-soluble.

2. The protein as claimed in claim 1, further comprising the amino acid sequence of GST.

3. An osteoclast differentiation inhibitor for inhibiting differentiation of a precursor cell of osteoclast into an osteoclast, comprising the protein as claimed in claim 1.

4. An osteoclast differentiation inhibitor for inhibiting differentiation of a precursor cell of osteoclast into an osteoclast, comprising the protein as claimed in claim 2.

5. An inflammatory bone resorption therapeutic agent for treating inflammatory bone resorption, comprising the protein as claimed in claim 1.

6. An inflammatory bone resorption therapeutic agent for treating inflammatory bone resorption, comprising the protein as claimed in claim 2.

7. A gene which codes a protein comprising the amino acid sequence of an extracellular region of Ror2 and being water-soluble.

8. The gene as claimed in claim 7, wherein the protein comprises the amino acid sequence of GST.

9. A recombinant vector comprising the gene as claimed in claim 7.

10. A recombinant vector comprising the gene as claimed in claim 8.

11. A method of manufacturing a protein, comprising the steps of:
causing expression of a gene coding a protein comprising the amino acid sequence of an extracellular region of Ror2 and being water-soluble in a microorganism so as to synthesize the protein and;
extracting the protein from the microorganism into water or an aqueous solution.

12. A method of inhibiting osteoclast differentiation, comprising the step of using the protein as claimed in claim 1.

13. A method of inhibiting osteoclast differentiation, comprising the step of using the protein as claimed in claim 2.

14. A method of treating inflammatory bone resorption, comprising the step of using a protein as claimed in claim 1.

15. A method of treating inflammatory bone resorption, comprising the step of using a protein as claimed in claim 2.
